(19) United States
(12) Patent Application Publication
(10) Pub. No.: US 2026/0363677 A1
(43) Pub. Date: Nov. 19, 2026

(54) MENSTRUAL PAD COLLECTION SYSTEM

(71) Applicant: Qurasense, Inc., Menlo Park, CA (US)

(72) Inventors: Brian WEINBERG, Redwood City, CA (US); Dan SIVILICH, Ocala, FL (US); Soren THERKELSEN, Copenhagen (DK); Sara NASERI, Aarhus (DK)

(21) Appl. No.: 19/153,728

(22) PCT Filed: Feb. 5, 2024
(86) PCT No.: PCT/US2024/014378

§ 371 Date: Aug. 5, 2025

Related U.S. Application Data
(60) Provisional application No. 63/443,940, filed on Feb. 7, 2023.

(51) Int. Cl. A61B 10/00; A61F 13/15; A61F 13/472; A61F 13/84

(52) U.S. Cl. CPC A61B 10/0045; A61F 13/472; A61F 13/84; A61B 2010/0074; A61F 2013/15243; A61F 2013/47281

(57) ABSTRACT

A system in which menstrual blood and vaginal discharge is collected for the purpose of diagnostics and health monitoring, more particularly to a system in which absorbent materials, such as feminine hygiene pads and urinary pads, are provided with strips or patches for collecting bodily fluids, containers that include desiccants are provided for transport and storage of the collected samples to and while at laboratories for analysis of Dried Menstrual Samples (DMS), Dried Menstrual Plasma Samples (DMPS), Dried Blood Spots (DBS), and/or Dried Plasma Spots (DPS), or some of which containers without desiccants are capable of maintaining adequate hydration to keep the collected cells intact for analysis using method such as DNA testing, slide based analysis, or cell cultures, various collection strips structures are provided that can be used for menstrual blood and vaginal discharge as well as for collecting and/or separating venous and capillary blood, and functional pad design elements to aid in sample collection, retention of collection strips, and to facilitate high-speed manufacturing of the pads.

### CROSS-REFERENCE TO RELATED APPLICATIONS

Priority is claimed on Provisional Patent Application No. 63/443,940, filed Feb. 7, 2023, the contents of which are incorporated herein by reference.

### STATEMENT REGARDING FEDERALLY SPONSORED RESEARCH OR DEVELOPMENT

Not Applicable

### REFERENCE TO A “SEQUENCE LISTING”, A TABLE, OR A COMPUTER PROGRAM LISTING APPENDIX SUBMITTED ON COMPACT DISC

Not Applicable

### BACKGROUND OF THE INVENTION

### 1. Field of the Invention

The present invention relates to a system in which menstrual blood and vaginal discharge are collected for the purpose of diagnostics and health monitoring, more particularly to a system in which absorbent materials, such as feminine hygiene pads and urinary pads, are provided with strips or patches for collecting bodily fluids. Transport containers that include desiccants or sample transport media are provided for storage of the collected samples, so sample types such as Dried Menstrual Samples (DMS), Dried Menstrual Plasma Samples (DMPS), Dried Blood Spots (DBS), and/or Dried Plasma Spots (DPS), can be mailed to a laboratory for sample analysis using a wide range of laboratory methods. As transport and storage containers can include both desiccants to keep samples dry, or liquids to maintain hydration or cell viability, for a range of laboratory analysis methods and targets including RNA sequencing, qRT-PCR), ctDNA testing, cfDNA, Enzyme-linked immunosorbent assay (ELISA), cell culture growth and analysis, Immunohistochemistry, Flow cytometry, Mass spectrometry, Nuclear magnetic resonance (NMR) spectroscopy, gas/liquid chromatography, PCR, Microarray analysis, Metabolomics profiling, Cell culture assays, Crystallography, Electrophoresis, Spectrophotometry and Microscopy. Included are also functional pad design elements to aid in sample collection, retention of collection strips, and the facilitation of high-speed manufacturing.

### 2. Description of Prior Art Including Information Disclosed Under 37 CFR 1.97 and 1.98

In the past, the only way to access systemic blood for diagnostic analysis was through invasive procedures such as a blood draw using a syringe, or a finger prick stick. For endometrial tissue analysis or samples from the vaginal tract, a biopsy or a scrape from the cervix or endometrium was necessary, both are invasive procedures and an uncomfortable experiences. Although it was known that both vaginal samples, endometrial tissue and systemic blood contain important biomarkers used for diagnostics in women health, known collection methodologies for collecting samples for testing were inconvenient, costly and time consuming.

This all changed with the introduction of a menstrual sample collection system which could be integrated into feminine hygiene pads and urinary pads. Collection devices such as strips could be provided in the pads and used to collect bodily fluids. The strips with the collected fluids could then be transported to laboratories for analysis. The collection of menstrual samples for analysis was no longer invasive or uncomfortable.

In this system, the absorbent pads may be formed with a fluid pervious top face sheet; a fluid absorbent layer, a fluid impervious back sheet, and a collection device such as a collection strip disposed between the top face sheet and the fluid absorbent layer. The fluid collection strip may be situated in a recess or pocket in fluidic contact with the absorbent layer. The recess or pocket is provided with an opening sized to allow the removal and/or insertion of the collection strip. The collection strip may be semi rigid and formed with a grippable pull tab portion extending from an edge of the strip for easy removal from the pad.

The collection strip is typically inserted through a slit in the top face sheet or through a gap in the end seal. The end seal may be made with an adhesive and/or mechanical crimp. A section of the end seal is left unsealed to facilitate an opening for the insertion of a strip, alternatively, a slit is formed during application of the top face sheet. Both manufacturing procedures of the pad result in reduced manufacturing speeds and also result in a slow and difficult insertion process of the strip during assembly due to the high drapability or limpness of the end seal.

After sample collection, once the collection strip is removed from the pad, it may be placed in a container for transport to a laboratory for analysis and storage. Dried Menstrual Samples (DMS), Dried Menstrual Plasma Samples (DMPS), Dried Blood Spots (DBS), and Dried Plasma Spots (DPS) require desiccation during transit to preserve them and prepare them for analysis in the lab. Other samples include live cells which must be kept hydrated during transit to a laboratory for analytical testing using laboratory analytical methods and targets such RNA sequencing, qRT-PCR), ctDNA testing, cfDNA, Enzyme-linked immunosorbent assay (ELISA), cell culture growth and analysis, Immunohistochemistry, Flow cytometry, Mass spectrometry, Nuclear magnetic resonance (NMR) spectroscopy, gas/liquid chromatography, PCR, Microarray analysis, Metabolomics profiling, Cell culture assays, Crystallography, Electrophoresis, Spectrophotometry and Microscopy. Depending upon the type of analysis being performed, special containers may be required for transport.

### BRIEF SUMMARY OF THE INVENTION

One aspect of the present invention relates to the structure of the absorbent pad. The pad includes a fluid pervious top face sheet, a fluid absorbent layer, and a fluid impervious back sheet sealed together along the peripheral edge. A pocket is formed between the top face sheet and the fluid absorbent layer. A fluid collection device is positioned in the pocket to collect bodily fluids for analysis. The collection device includes a pull tab extending from the collection device body which can be gripped by the user to facilitate removal of the collection device. The pad includes a notch aligned and slightly overlapping the pocket extending inwardly from the peripheral edge of the pad such that the pull tab of the collection device is exposed.

The pad preferably includes an oval (or other shape) indicator printed on the top face sheet which is centered under the vaginal canal. The oval assists in correct positing the pad pocket during use and is sized such that the time it takes the user to fill the oval with fluid corresponds to the time it takes to complete the fluid collection by the fluid collection device.

The pad may also have a direction indicator on the top face sheet proximate the notch. This indicator informs the user of the correct direction of the pad when installing in their undergarment for use.

Another aspect of the present invention relates to a container provided for transporting a fluid collection device of the type utilized in an absorbent pad to collect a sample or in the absents of an absorbent pad after a liquid sample has been applied to the strip directly, such samples could be menstrual sample collected in a cup or from a tampon, venous blood collected using venipuncture, or capillary blood obtained using a lancet. The container preferably includes a slot adapted to receive the fluid collection device, and a desiccant. The container includes a channel connecting the slot and the desiccant to allow vapor transfer from the fluid collection device for dehydration of the sample during transit.

Another aspect of the present invention relates to a strip sample container which be manufactured in a way where the container can be molded as a unified structure.

Another aspect of the present invention relates to a collection strip for use in an absorbent pad to collect fluid for analysis. The strip can also be used in a direct fashion by manually dosing the collection paper with a liquid sample such as one collected using a menstrual cup or tampon, a venous draw or capillary finger prick, or a urine sample while the collection strip is resting on a surface. The collection strip is flexible and includes a collection paper, a thin plastic base film and a bottom frame. The collection paper is mounted to the thin plastic base film using the bottom frame. The bottom frame may be a single sided medical tape coated with an adhesive.

Another aspect of the present invention relates to a plasma collection strip provided for use in an absorbent pad to collect plasma samples. The strip can also be used in a direct fashion by manually dosing the collection paper, filter surface, and/or input port with a sample in liquid form, such as one collected using a menstrual cup or tampon, a venous draw or capillary finger prick, or a urine sample, while the collection strip is resting on a surface. The collection strip includes one or more collection papers and at least one filter. The filter provides filtration of the blood and isolates a collection paper from the unfiltered blood. The collection paper can receive filtrate across the entirety of its surface from the filter, functioning solely for collection or can receive whole blood or filtrate on the end portion of said paper, so the collection paper also performs lateral blood separation as the fluid moves through its lattice structure. In addition, the filter can be incorporated into a layer of the strip that the user removes after collection, facilitating faster drying of the collection paper and reducing the likelihood of hemolyzed blood transferring to the collection layer during desiccation.

In one preferred embodiment of the plasma collection strip, the filter includes a single layer of track-etch (TE) membrane. The collection paper is situated between the TE membrane and the base film. The TE membrane may be torn off after collection by the user to facilitate faster drying of the collection paper and to reduce the likelihood of hemolyzed blood transferring to the collection layer during desiccation.

In another preferred embodiment of the plasma collection strip, the filter includes an irregular path filter formed in a sandwiched layer of medical tape.

In another preferred embodiment of the plasma collection strip, the filter includes a tortuous path depth type filter alone or used in tandem with a track-etch membrane as the primary filter.

Another aspect of the invention relates to a cell collection strip for use in an absorbent pad. The strip is used for collecting live cell samples and for returning the cells to the laboratory in a hydrated state for laboratory analysis. The strip includes a base film and an adhesive frame and a cell collection medium. The cell collection medium is situated between the base film and an adhesive frame. The film can include openings in the film to allow fluid to flow through the strip. A wicking collection medium layer can include materials such as a foam, lattice, or fiber or combination of the latter whose function is for trapping cell material when the collection device is being used. A secondary mesh layer can be incorporated to increase cell capture.

Another aspect of the invention relates to a cell collection patch for use on an absorbent pad top sheet layer. An absorbent pad can include top sheet that blocks cellular material from reaching an embedded strip. A patch resided on top of the top sheet which allows direct capture of menstrual flow without the menses being absorbed and/or filtered by the pads top layer. The patch captures the cellular sample and is used for returning the cells to the laboratory in a hydrated state for analysis.

The patch preferably includes a frame with surfaces and edges that are safe and comfortable to be in contact with the woman vulva and surrounding epidermis. It is constructed using soft compliant materials. The patch can include a wicking layer and a mesh layer. The mesh layer is situated below the wicking layer to capture additional cells, the mesh size can be adjusted depending on the requirements of the analysis being performed. The patch surface has multiple input ports. The collection layers trap the cells to be processed at the lab with most of the liquid soaking through to the pad, such that sample collection time can be extended to capture more cellular material.

Another aspect of the present invention relates to a DMS or DBS collection strip that utilizes a double-sided medical tape or transfer adhesive for adherence of the collection paper to the base film.

Another aspect of the present invention relates to a method of high-speed manufacturing of absorbent pads, such as feminine hygiene pads and urinary pads, wherein the pocket in the pad is created by zone coating the pad with hot melt adhesive to secure the nonwoven facing material to the pad and to form a contrived impervious zone (or zones) around the pocket.

### BRIEF DESCRIPTION OF THE SEVERAL VIEWS OF DRAWINGS

FIG. 1 is a perspective view of the absorbent pad of the present invention showing the notch cut in the peripheral edge, and printed indicators, an oval and the directional arrow in this case.

FIG. 2 is an elevational view of the pad of FIG. 1 showing the pull tab of the collection strip extending into the notch.

FIG. 3 is an exterior view of a container for sample transport with a cut away to show a portion of the interior thereof.

FIG. 4 shows the container with the lid open and collection strips partially inserted into the container.

FIG. 5 is another view of the container with the lid open.

FIG. 6 is an exploded view of the container with the lid open showing the interior with a single collection strip in place.

FIG. 7 is a cut away view showing the interior of the container.

FIG. 8 is a perspective view of the interior of the container.

FIGS. 9A and 9B are views of the tether and the triple lip seal.

FIGS. 10A, 10B, 10C and 10D are views of a cell collection container for strips of samples that do not require desiccation that can be filled with liquid such as a buffer.

FIG. 11 shows the exterior of a cell collection container for collection patches that do not require desiccation.

FIG. 12 is an exploded view of the container of FIG. 11.

FIG. 13 shows the container of FIG. 11 with the lid partially open to expose the interior with the patch inside.

FIGS. 14A-14D and 15A-15B show a second preferred embodiment of a strip sample container.

FIG. 16 is an exploded view of a DMS or DBS collection strip.

FIG. 17 is an exploded view of a plasma collection strip that uses a track-etch membrane as a primary filter that also incorporates DMS or DBS collection.

FIG. 18 is an exploded view of a plasma collection strip that uses a depth pre-filter adhered to a removable layer using double sided tape or a transfer adhesive that also incorporates DMS or DBS collection.

FIG. 19 is an exploded view of a multi-stage plasma filter collection strip which includes a depth pre-filter and a track-etch membrane adhered to a removable layer using double sided tape or a transfer adhesive that also incorporates DMS or DBS collection.

FIG. 20 is an exploded view of another multi-stage plasma filter collection strip which includes a heat staked depth pre-filter and a Track-etch membrane filter adhered to a removable layer that also incorporates DMS or DBS collection.

FIG. 21 is an exploded view of a lateral flow plasma collection strip in which there is no pre-filter, whose removable layer includes a sample input port, that also incorporates DMS or DBS collection.

FIG. 22 is an exploded view of a plasma collection strip track-etch membrane pre-filter adhered to a removable layer with a Lateral Flow collection layer that also incorporates DMS or DBS collection.

FIG. 23 is an exploded view of a plasma collection strip with a depth pre-filter adhered to a removable layer using double sided tape or a transfer adhesive with a Lateral Flow collection layer that also incorporates DMS or DBS collection.

FIG. 24 is an exploded view of another multi-stage plasma filter collection strip which includes TE membrane pre-filter and an asymmetrical path pre-filters adhered to a removable layer with a lateral flow collection layer that also incorporates DMS or DBS collection.

FIG. 25 is an exploded view of a cell collection strip designed to slide into a strip pocket.

FIG. 26 is an elevation view of a patch type cell collector on a pad.

FIG. 27 is an exploded view of the patch type cell collector of FIG. 26

FIGS. 28A, 28B and 28C show a DMS or DBS collection strip that uses a double-sided tape or transfer adhesive to adhere the filter paper to the base film.

FIG. 29 is a top of a partially manufactured pad with the top layer removed.

FIG. 30 is a structural view of the hot melt adhesive slot coating nozzle and a sectional view of the pad.

FIG. 31A shows a portion of the pad and the fluid being absorbed directly into the absorbent layer. 31B shows a portion of the pad outside of the pocket with adhesive zones that provide a controllable barrier by increasing the add-on level of the adhesive causing the fluid to disperse in the non-woven top layer.

### DETAILED DESCRIPTION OF THE INVENTION

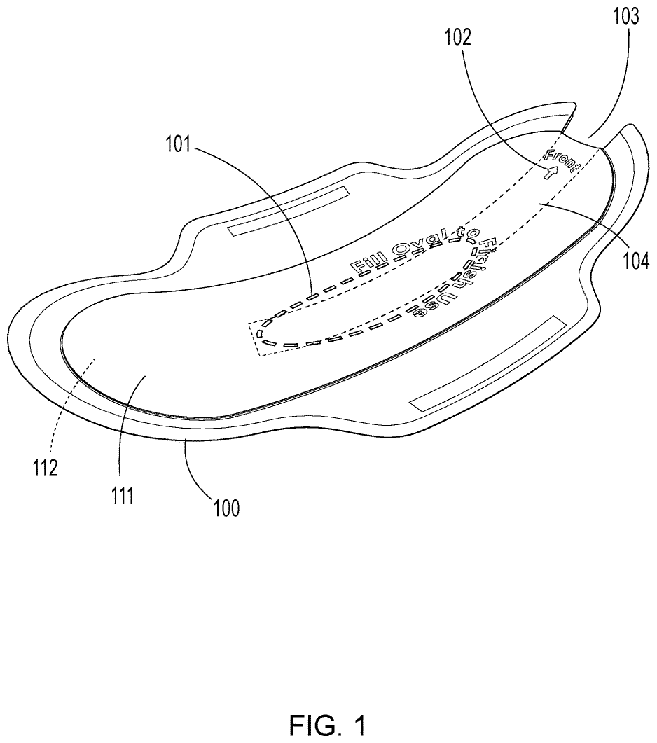

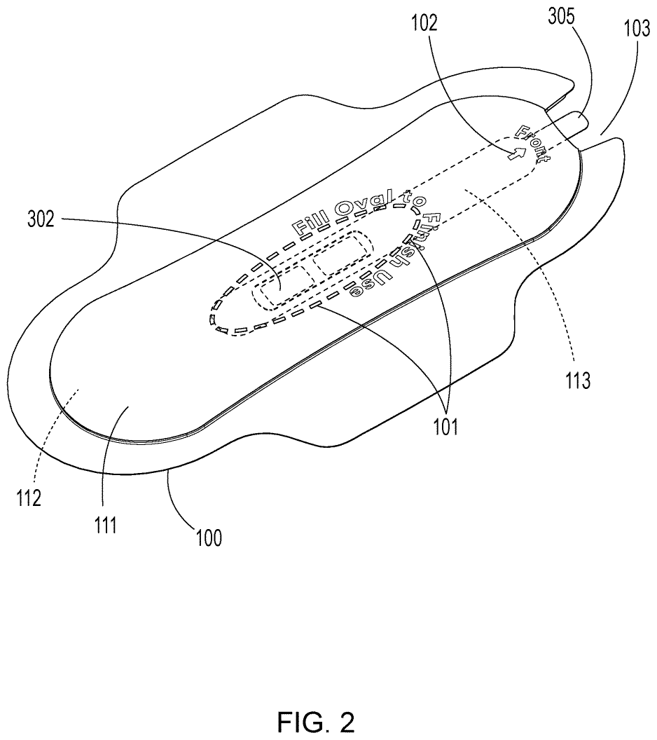

FIGS. 1 and 2 show the menstrual pad (100) of the present invention. Pad (100) includes a fluid pervious top face sheet (111) and a fluid absorbing layer (112). It also includes a pocket 104 formed between the top face sheet and fluid absorbing layer. The pocket is designed to receive a removable fluid collection device (113).

The fluid collection device (113) may take the form of a semi rigid collection strip (301) which is inserted in the pad pocket to collect bodily fluids for analysis. The collection strip may include one or more collection papers (302). Pocket (104) retains the collection strip and prevents it from migrating from its position with the collection paper(s) centered under the vaginal canal. The pocket may be formed by selectively applying construction adhesive between the sheets that make up the pad.

As seen in the figures, the contour of the pad is provided with a recess or notch (103) cut through the totality of pad layers or partially through the pad, piecing selective layers of the pad starting from the top or bottom of the pad. Notch (103) cut overlaps the edge of the fluid absorbing layer which allows access to the pocket (104) and the strip pull tab (305) when it is seated in the pocket. The notch is formed in the final die cut of the pad during manufacturing using a modified rotary die cutter.

An oval (101) is preferably printed on top face sheet (111) of the menstrual pad. Oval (101) is centered under the vaginal canal. The oval is sized so that the time it takes for the user to fill the oval with fluid corresponds to the time it takes to complete the collection onto the collection paper(s) (302). The oval (101) can also be shaped any circular or rectilinear or graphical object depending on the requirements of the particular collection strip that will be used with the pad. The oval also provides a reference point to indicate where the collection paper(s) (302) of the strip are located to ensure the user knows where to direct their menstrual flow relative to the position of the pad. The oval (101) may also be sized and shaped in such a way, that it can support a visual estimation of the amount of sample collected in the pad.

A pad direction indicator (102), preferably including the word “Front” and an arrow, is preferably printed on the top face sheet close to and in line with notch (103) of the pad in order to inform the user that the pad is directional, unlike most standard pads that can be used with either end forward.

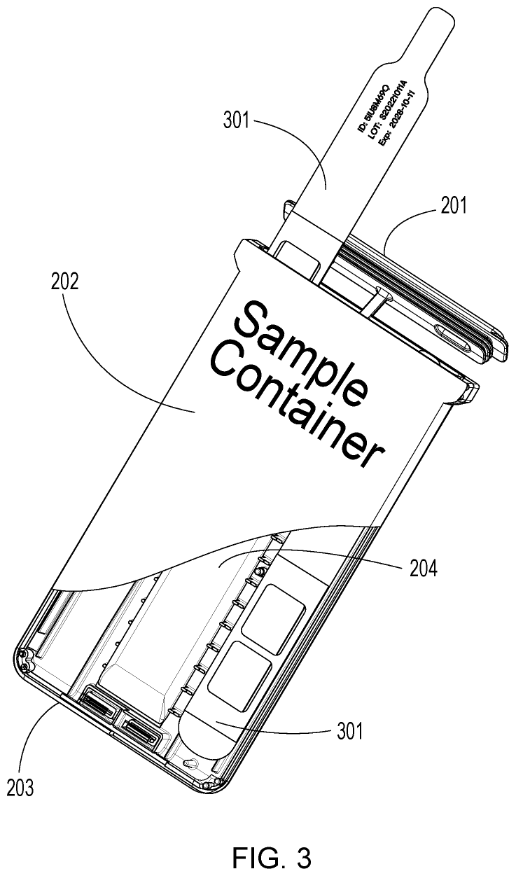

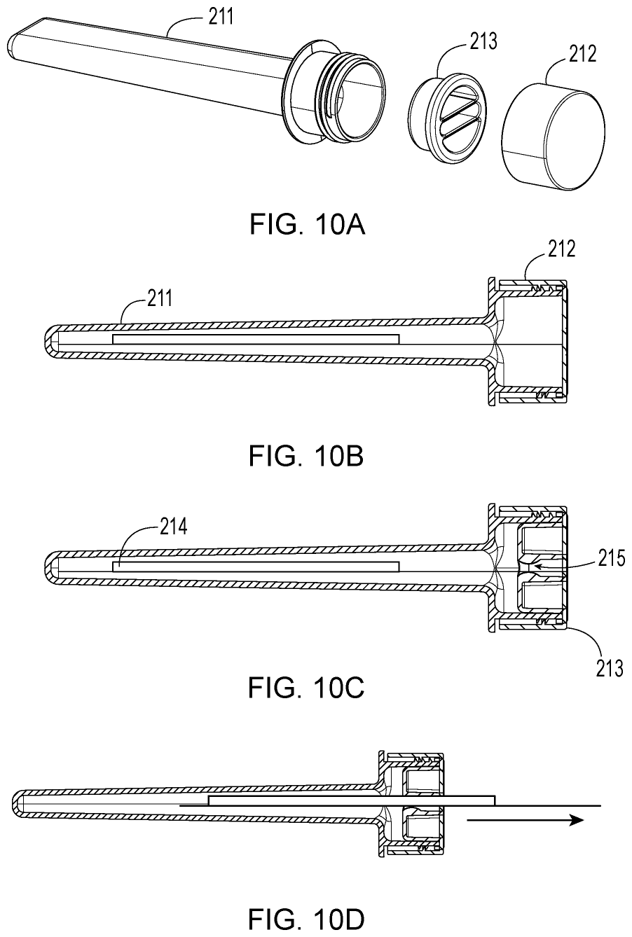

FIGS. 3 through 13 illustrate containers used to protect samples during transport and storage. FIGS. 3 through 9B relate to containers which desiccate the samples to preserve them and prepare them for analysis in the lab. FIGS. 10 through 13 illustrate containers for cell collectors which do not require desiccation during transport instead are optimized to maintain hydration.

As illustrated in FIGS. 3 through 9B, the container is formed of case halves (203) which are injection molded and mirror images, so one component can be used for both halves. There are two slots (208) designed to receive the collection strips (301). Each slot (208) has features (206) designed to guide the strips into position but still allow for efficient dry-down by allowing vapor transfer from the strip to the desiccant by way of channels designed into the guides (207). The case halves (203) are held together primarily using pins (205) with a wraparound sticker (202) used for redundancy.

An elastomeric lid (201) with a triple lip seal (210) is designed for easy user removal and closure while providing sufficient sealing to reduce the rate of vapor migration from the environment to an acceptable level. Vapor transmission rate is also reduced by the wrap-around stickers (202). Lid (201) is connected to the case by a molded-in tether (209).

The sample container houses a molecular sieve desiccant (204) whose function is to dry the samples in a consistent and predictable manner and then maintain the desiccated state during transport and while the strips are being processed in the lab. The desiccant (204) specification includes enough desiccating capacity for two strips (301), which may be of any type, so can be dried down and maintained in a desiccated preserved state for an extended period.

An oxygen absorber (not shown) may also be included in the container. The oxygen absorber further preserves the sample by reducing its oxidation rate.

A poly sticker (202) is wrapped around the case after the container is assembled to serve as a secondary closure method, to reduce the vapor transmission rate, and is used for labeling with graphical instructions, warning, branding, and/or limitations.

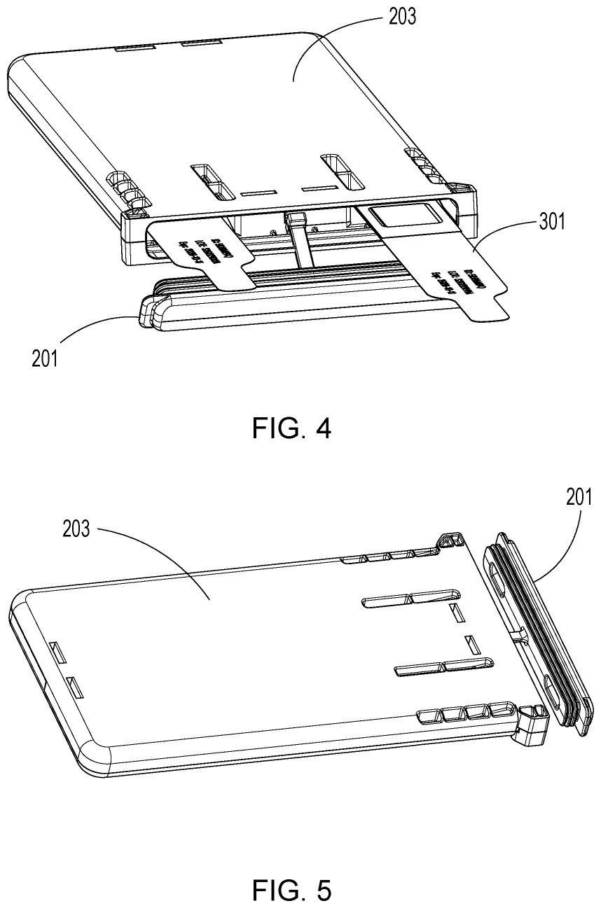

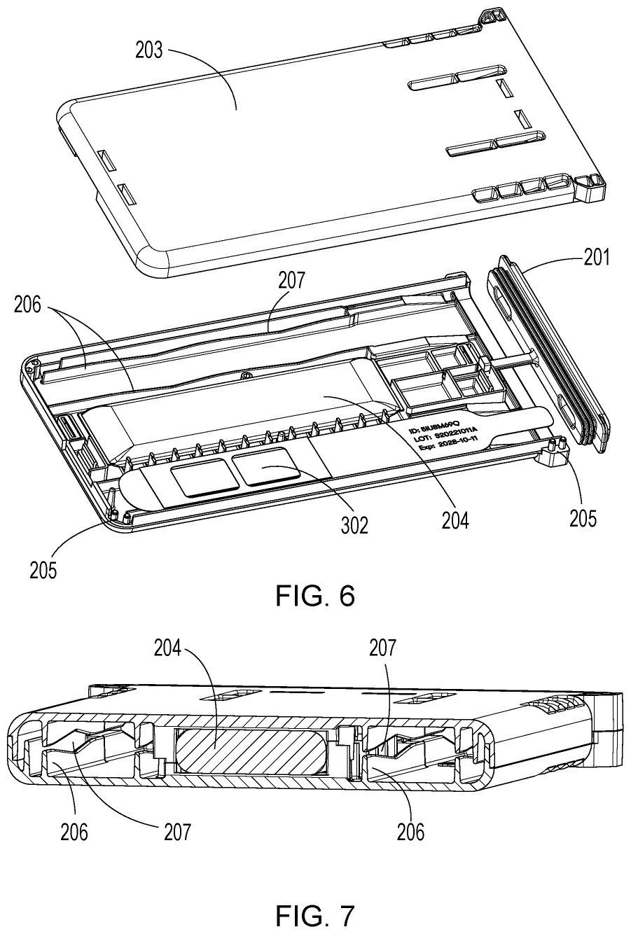

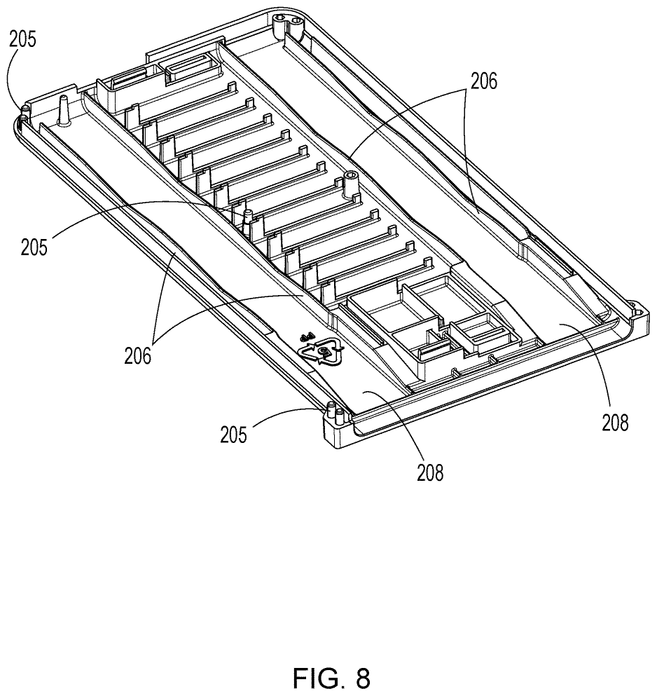

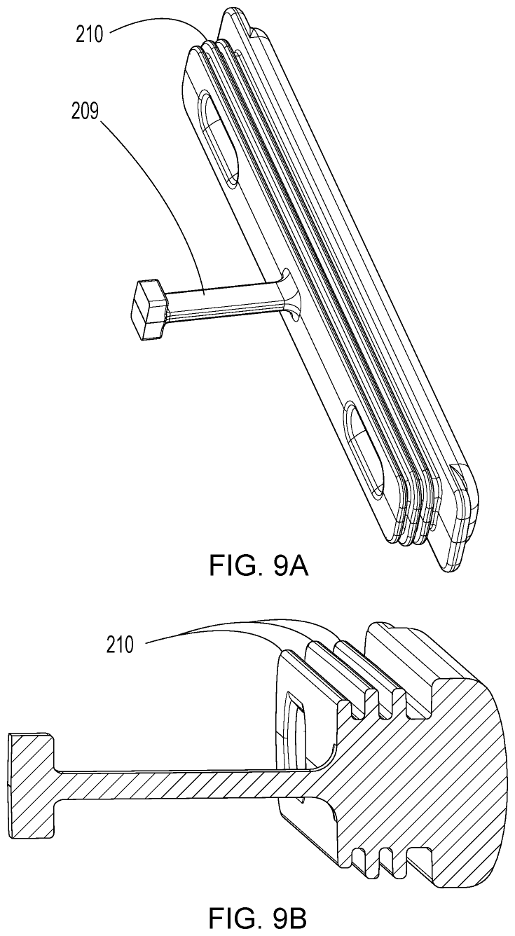

FIG. 3 shows lid (201), collection strips (301), the wrap around sticker (202), case half (203) and desiccant (204). FIG. 4 shows lid (201), collection strip (301) and case half (203). FIG. 5 shows lid (201) and case half (203). FIG. 6 shows collection paper (302), lid (201), case half 203, desiccant (204), pins (205), guides (206), and vapor flow channels (207). FIG. 7 shows desiccant (204). Guides (206), and vapor flow channels (207). FIG. 8 shows pins (205), guides (206) and slots (208). FIGS. 9A and 9B show tether (209) and triple lip seal (210).

FIGS. 10A through 10D illustrate another embodiment of a return cell sample container (211) which can be used for samples that do not require desiccation. This variation is water and airtight to maintain the state of the sample. In some cases, the container may contain a buffer solution that will be used to suspend the sample and in other cases no buffer will be included so the sample will remain in the same state as it was when collected. In both cases, the sample container can be used for transportation and preservation of the sample.

As illustrated in these figures, the container body (211) is an injection molded component designed to both protect the sample and maintain its state. It is shaped to minimize the amount of buffer solution needed to maintain sample quality.

The container lid (212) provides an airtight and watertight seal to prevent ingress and leakage. An extraction insert (213) is optionally used by the lab when extracting the strip from the container. It has compression features (215) designed to “wring” out the collection material (214) as the strip is removed.

FIG. 10A through 10C show container body (211), extraction insert (213), container lid 212, cell collection strip 214, collection strip foam and the extraction insert compression feature 215, see FIG. 10C. FIG. 10D shows the extraction insert compression features (215) which “squeezes” the foam during removal forcing sample out of the material into liquid.

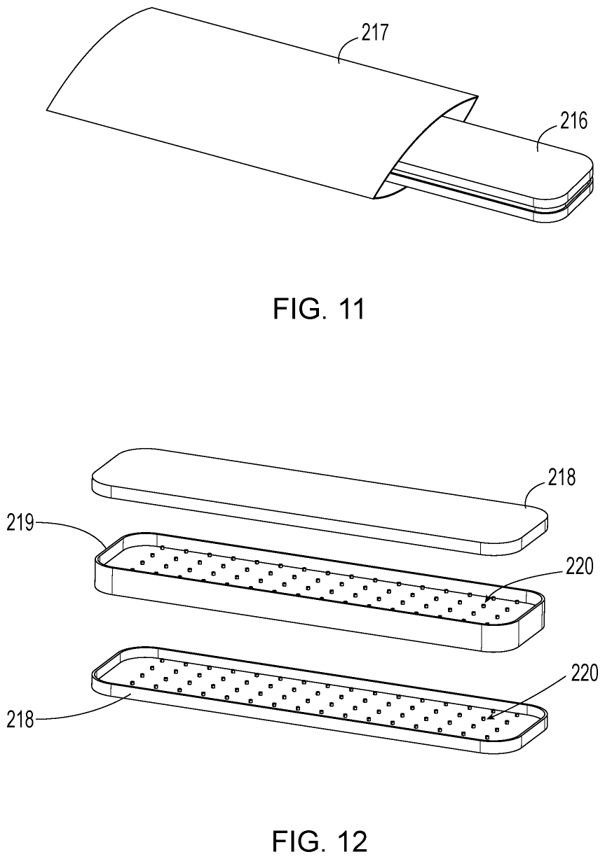

FIGS. 11, 12 and 13 illustrate another embodiment of the return cell collection sample container (216) for collection patches (402) that do not require desiccation. This container is designed to be stored and shipped in a vapor barrier pouch (217). The combination of the container and pouch is designed to maintain the samples in their collected state for extended periods during transportation.

Container (216) includes a container body (219) which is an injection molded component whose primary function is to protect the sample(s). The surface on which the patch (402) rests consists of a plurality of spacer pegs (220) which keep the sample off the flat surfaces so sample integrity is maintained during storage and transport.

The container lid(s) (218) are made from elastomeric material. They seal tightly around the outer surfaces of the container body (219). The lids also have spacer pegs (220) to keep the patch off the flat surfaces.

The return/storage pouch (217) may be provided in order to keep the sample hydrated and cells alive. The cell sample container (216) is stored and shipped in a vapor barrier pouch (217).

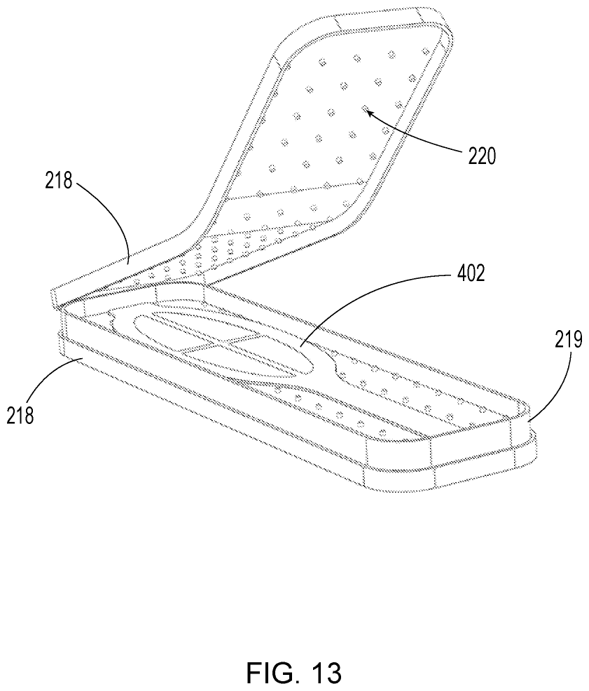

FIG. 11 shows a sample return container for cell patches (216) and the vapor barrier pouch (217). FIG. 12 shows the cell sample container lid 218, the cell sample container body (219) and spacer pegs (220). FIG. 13 shows the cell sample container lid (218), the cell sample container body (219), spacer pegs (220) and the cell collection patch (402).

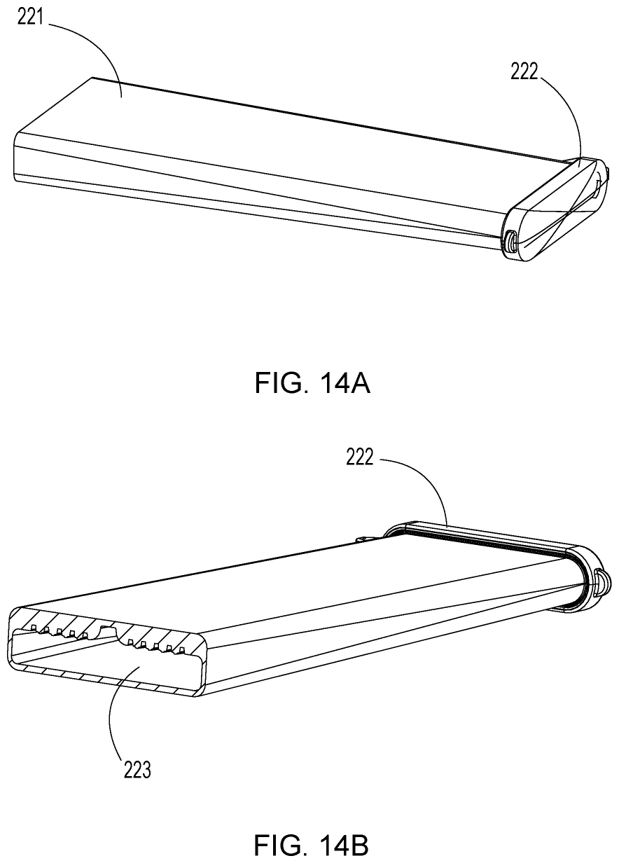

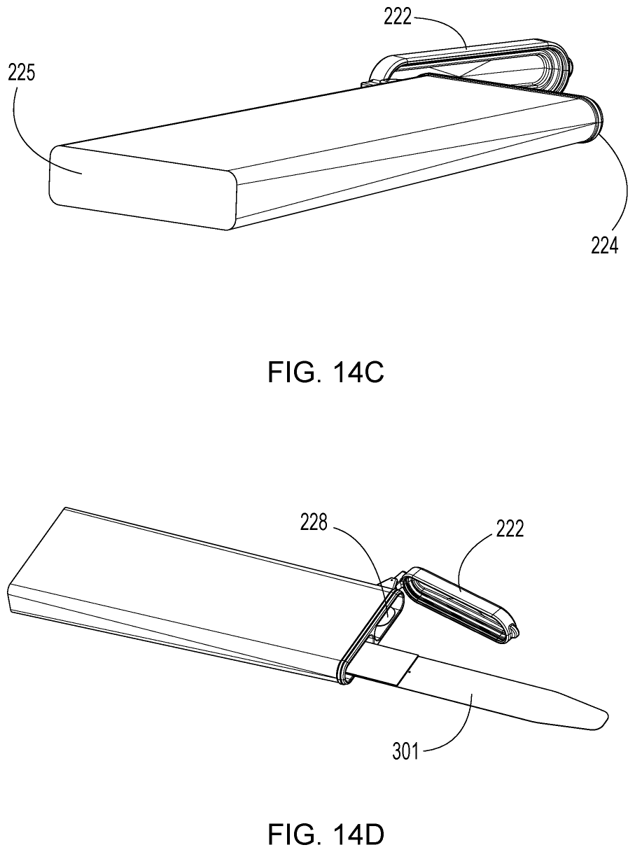

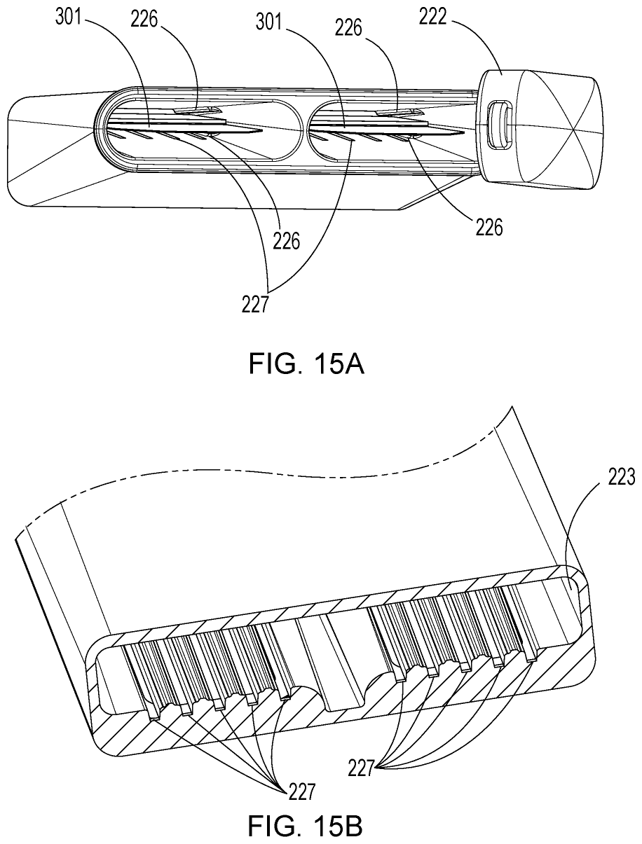

FIGS. 14A-14D and 15A-15B illustrate another embodiment of a strip sample container designed to store and desiccate samples. This strip sample container can be molded as a unified structure for production and assembly efficiency.

To mold as a single component, the case (221) is designed with deep draws from both longitudinal directions. One draw forms the strip cavity (228) and the other forms the desiccant cavity (223). The strip cavity is molded in such a way to have features (226) seen in FIG. 15A that prevent the collection paper zone where samples will be punched from touching the interior of the case. To allow for vapor transfer from the collection paper to the desiccant a series of slots (227) as seen in FIG. 15B are molded into the case. These slots (227) are located directly over the collection paper and are of a sufficiently small size to prevent desiccant from passing into the strip cavity.

A lid (222) is molded into the end of the strip cavity as part of a case using a living hinge that allows the user to easily access the strip slots. In place of a molded in living hinge, the lid (222) can be a separate component made from the same or a dissimilar plastic which assembles to the case (221) using a pin, sliding mechanism or other similar mechanism. The lid (222) also functions to seal the interior of the case from the environment using a snap fit feature (224) which has the effect of extending the life of the desiccant when the case is not inside a vapor barrier pouch.

During assembly small desiccant balls and optionally an oxygen absorber packet are loaded into the desiccant cavity (223) and then sealed inside the cavity using a foil and/or plastic film (225). This film can be adhered by using adhesive and/or other techniques such as ultrasonic welding.

FIG. 14A shows the molded case (221) and snap lid (222). FIG. 14B shows the snap lid (222) and desiccant cavity (223). FIG. 14C shows the open snap lid (222), lid snap feature (224) and desiccant cavity film seal (225). FIG. 14D shows strip slot (228), lid (301) and collection strip (301).

FIG. 15A shows lid (222), contact prevention features (226), vapor transmission slots (227) and strip (301). FIG. 15B shows slots 227.

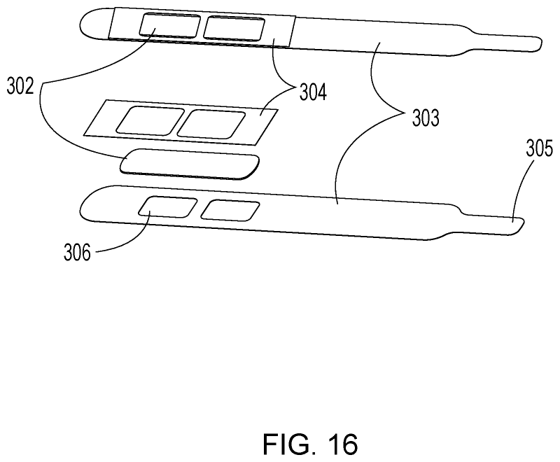

FIGS. 16 through 28 show collection strips of various constructions. FIG. 16 illustrates a DMS or DBS collection strip. To collect samples, the strip (301) includes a single collection paper (302). Collection paper (302) is mounted to a thin plastic base film (303) with the bottom frame (304). The bottom frame (304) is a single sided medical tape coated that uses an inert acrylic (or equivalent) medical grade adhesive. Pull tab (305) is used during the assembly process to insert the strip into the pad and by the user to remove the strip (301) from the pad after collection is completed. The unique device identification (UDI) (not shown) is printed onto the back of the strip or printed onto a durable sticker and adhered to the back of the strip during assembly. It includes a unique serial number, its LOT, and an expiration date. The bottom film (303) also includes multiple punch holes (306) to facilitate processing in the lab.

FIG. 16 shows the DMS or DBS collection strip (top) (301), pull tab (305), collection filter paper (302), bottom frame (304), punch holes (306) and the base film (303).

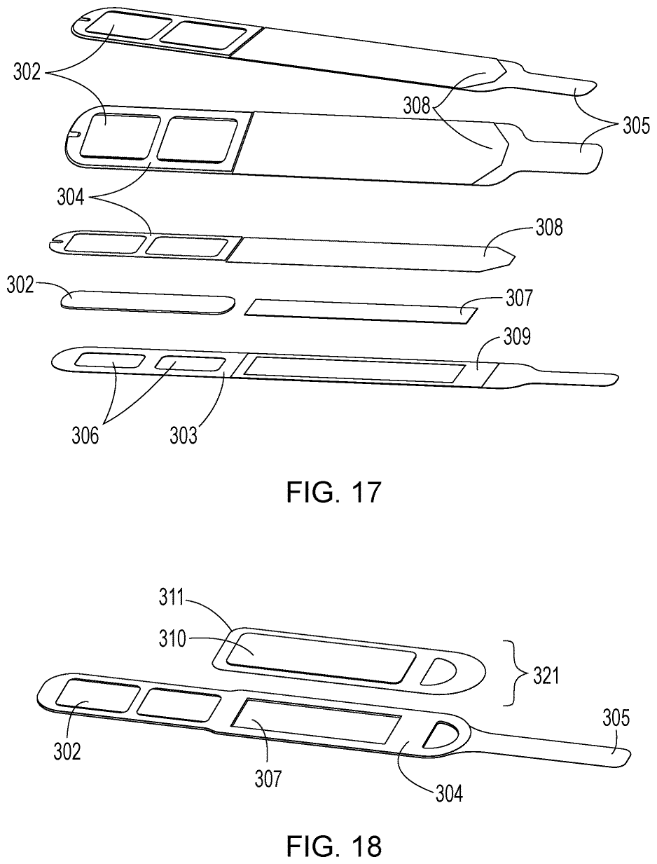

FIGS. 17 through 25 show collections strips that include a section that filters red blood cells from menstrual, venous, or capillary blood. This filter results in the generation of plasma which will be dehydrated in the sample container resulting in a Dried Menstrual Plasma Sample (DMPS) if the source sample matrix is menses or a Dried Plasma Sample (DPS) if the sample was venous or capillary blood. Separation can be performed using various configurations of depth, lateral flow, and/or membrane filters.

The configurations can be broken into three general descriptive groups: strips with throughflow filtration, strips that use a combination of throughflow and lateral flow, and strips that only use lateral flow filtration.

As seen in FIGS. 17 and 18, the throughflow strips use the plasma collection paper (307) only as a sample receptacle. The blood flows through a filter directly into the collection paper (307). The filter provides filtration and isolates the collection paper from whole unfiltered blood. This configuration has the advantage of accelerated collection times vs. other configurations because of the large surface area involved in filtering the blood sample. The filter stack uses one or more membranes that can be either track-etched fixed pore size filters and/or irregular tortuous path type filters.

As seen in FIG. 17, a single-stage blood separator with a track-etch membrane is shown. It is an embodiment of the filter for a DMPS or DPS strip. A single layer of track-etch membrane (308) is used to filter menstrual sample, venous, or capillary blood. The pore size of the membrane can be from 0.05 um to 2.0 um The collection paper (307) is sandwiched between the track-etch (TE) membrane (308) and the base film (304) with the TE membrane and film connected by a medical grade transfer adhesive or a double-sided tape (309) to retrieve and/or facilitate drying of the collection paper after use the membrane peels off or is cut away.

FIG. 17 shows the DMS or DBS collection paper (302), pull tab (305)., bottom frame 304, punch holes (306), bottom film (303), plasma collection paper (307), the Track-etch membrane 308 and the transfer adhesive (309).

FIG. 18 shows a depth pre-filter (Vivid, IPOC, Microglass fiber) strip which is another embodiment of the plasma system that uses an irregular tortuous path depth filter such as Vivid™ by Pall to perform blood separating functionality (310).

A caveat of depth filters is that they retain a volume of whole blood so they must be decoupled from the collection paper (307) during desiccation or hemolysate will contaminate the plasma sample. To decouple the filter, the depth filter is mounted on a removable peel-off layer that consists of a single-or double-sided medical tape (311). The depth filter (310) is discarded by the user after collection, before the strip is inserted into the sample container for storage and transport.

FIG. 18 shows the DMS or DBS collection paper (302), pull tab (305), plasma collection paper (307), depth filter 310, and the peel-off laminate (311).

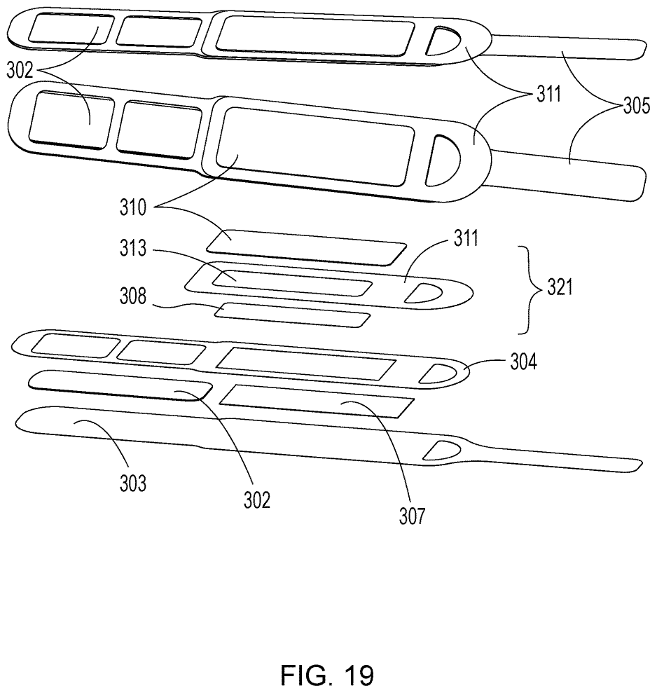

FIG. 19 shows a multi-stage plasma filter stack which includes an asymmetrical tortuous path pre-filter (Vivid, IPOC). The asymmetrical filter slows down migration of red blood cells (RBCs) through the filter structure, decreasing the quantity of RBCs that need to be filtered by the TE. This reduces the level of hemolysate in the final dried sample.

A depth pre-filter preceding a TE membrane in the fluid flow path facilitates greatly decreased red blood cell migration to the collection paper. A peel-off layer is constructed with a dual-lined double-sided tape (311). The adhesive layer on the top of the tape is used to adhere the depth filter (310) and the bottom adhesive layer is used to adhere the TE membrane (308). It also adheres the peel-off layer to the bottom frame (304) and seals off the plasma collection paper (307) from exposure to whole blood during collection (whether in a pad or when being dosed directly with a liquid menstrual, venous or capillary blood sample).

To establish a more robust fluidic connection between the pre-filter (310) and TE membrane (308), a PE drain membrane (313) can be included in-between the pre-filter (310) and TE membrane (308).

A depth pre-filter+Track-etch membrane primary filter can be used as an alternate embodiment. It uses a single sided tape (311) instead of a double-sided tape with the pre-filter heat staked (314) to the tape instead of using adhesive to join the materials.

FIG. 19 shows the DMS collection paper (302), pull tab (305), bottom frame (304), bottom film (303), plasma collection paper (307), Track-etch membrane (308), depth pre-filter (310), peel-off laminate (311), and optional PE drain membrane (313).

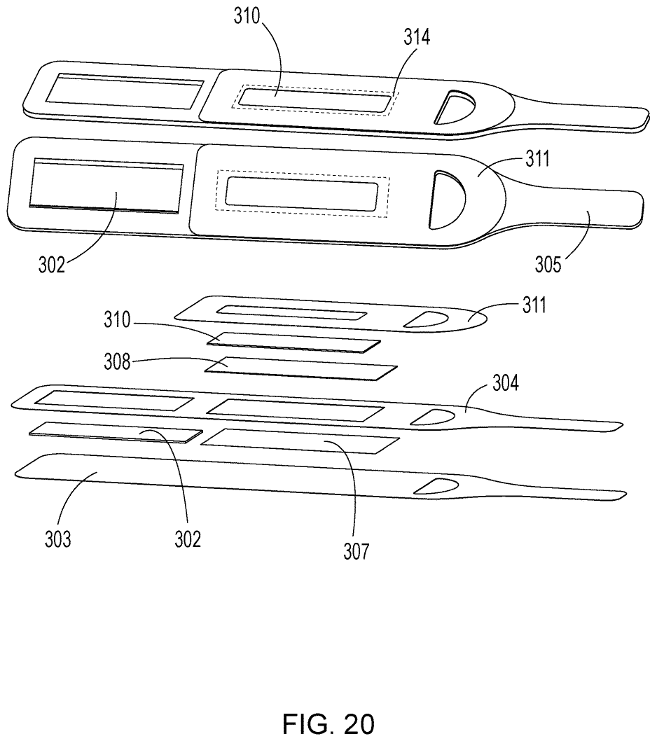

FIG. 20 shows the DMS or DBS collection paper (302), pull tab (305), bottom frame (304), bottom film (303), plasma collection paper (307), track-etch membrane (308), the depth pre-filter (310), peel-off laminate (311), and heat stake zone (314).

### Lateral Separator Configuration(s)

When Cytiva LF1 (or equivalent) is used as a plasma collection paper, additional functionality can be leveraged to perform blood separation. LF1 is constructed using microglass fiber and binder material. As blood flows laterally across the LF1, the glass fibers trap the relatively large RBCs, preventing them from migrating across the paper as the paper wicks the blood from input to the opposite end. The LF1 is essentially a whole blood collector and lateral tortuous path plasma separator in one. The LF1 is labeled with the direction of lateral flow separation in the following device images.

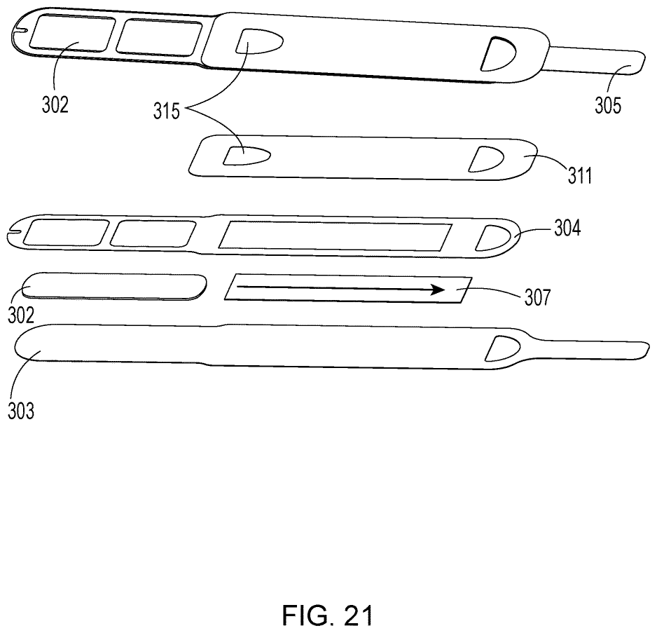

FIG. 21 illustrates the situation where there is lateral flow only (no pre-filter). As seen in this figure, the plasma separation portion of the strip is a rectangular piece of LF1 (or equivalent) (307) that is sealed off, with the peel of laminate (311), from exposure to whole blood except for a single input port (315). The port is shaped to decrease the amount of liquid exposed to the edges of the LF1 to prevent unfiltered liquid from bypassing the paper and instead wicking down the edges. The single sided tape (311) used to cover the LF1 can be removed after collection to simplify processing at the laboratory.

FIG. 21 shows the DMS or DBS collection paper (302), pull tab (305), bottom frame (304), bottom film (303), plasma separation and collection paper (307), peel-off laminate (311) and whole blood input port (315).

### Track-Etch Membrane Pre-Filter+Lateral Flow

To decrease the number of RBCs presented to the import port of the lateral flow collection paper (307), a pre-filter can be added to the input port (308). This embodiment uses a Track-etch membrane (308) as a prefilter.

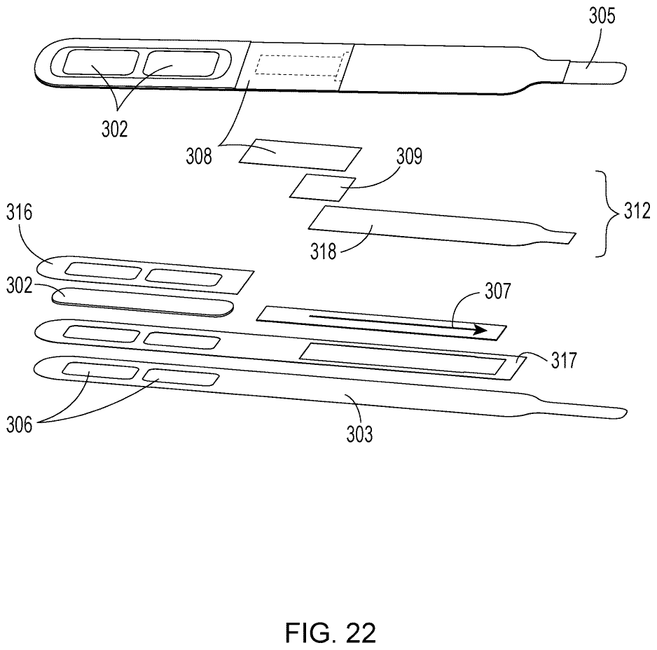

As seen in FIG. 22, this embodiment is constructed using a double-sided tape (317) sandwiched between plastic films (316, 318 and 303). The base film (303) has punch holes (306) under the DMS or DBS collection paper (302) to facilitate processing in the lab. The DMS or DBS paper (302) is held in place using a plastic film (316) also with punch holes. The lateral separating plasma collection paper (307) is mounted onto the top of the double-sided tape (317) and is encapsulated by a removable peel-off plastic film (318).

The input port of the plasma separating paper (308) is covered with a track-etch membrane (308) to pre-filter blood. A transfer adhesive (309) connects and seals the track-etch membrane (308) to the peel-off plastic film (318). The peel-off layer (312) is removed, it also includes the pre-filter (308), peel-off plastic film (318), and transfer adhesive (309) which are attached to the peel-off layer. This allows for improved sample desiccation, reduced contamination of the sample with hemolysate and efficient processing at the laboratory due to better access to collection paper. FIG. 22 shows the DMS or DBS collection paper (302), pull tab (305), punch holes (306), base film (303), plasma separation and collection paper (307), Track-etch membrane (308), transfer adhesive (309), peel-off layer (312), input port (315), plastic film (DMS or DBS side) (316), double sided tape (317) and removable plastic film (plasma side) (318).

### Depth Pre-Filter+Lateral Flow

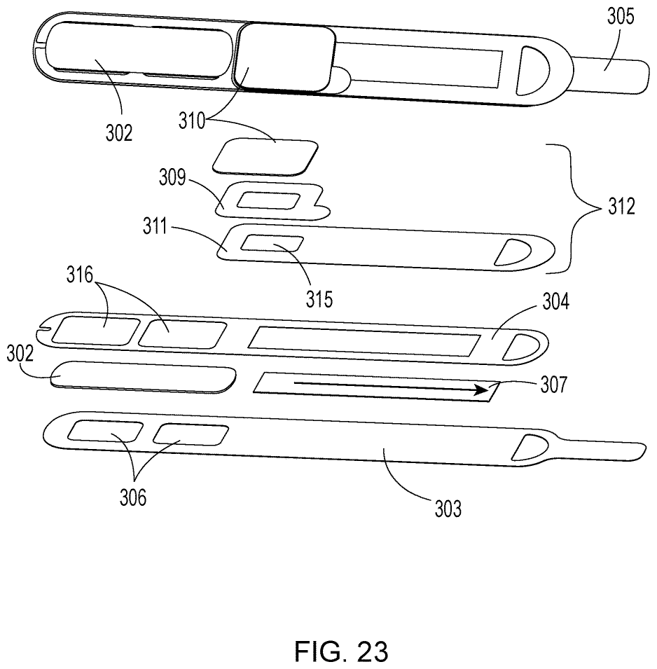

FIG. 23 illustrates the situation where a depth pre-filter (310) captures RBCs using a tortuous path that allows plasma to present itself at its bottom surface before RBCs migrate through. The filter can be asymmetrical such as Vivid™ or homogenous in structure, such as a glass microglass fiber filter. The lateral separating and collection paper (307) provides an additional tortuous patch for the RBCs as they flow down the paper effectively separating RBCs from plasma during the wetting of the strip. The peel-off layer (312) is removed by the user or laboratory to assist in drying the paper, reducing hemolysate contamination and to access it for processing.

FIG. 23 shows the DMS or DBS collection paper (303), pull tab (305), bottom frame (304), punch holes (306), base film (303), plasma separation and collection paper (307), transfer adhesive (309), depth pre-filter 310, peel-off laminate (311), peel-off layer (312) and input port (315).

22

### Depth Pre-Filter+Track-Etch Membrane Pre-Filter+Lateral Flow

Not shown is an alternative embodiment of the strip shown in FIG. 23 where a Depth pre-filter is followed by a TE membrane to increase filtering capabilities.

### Pre-Filter+Inertial Separator+Lateral Flow

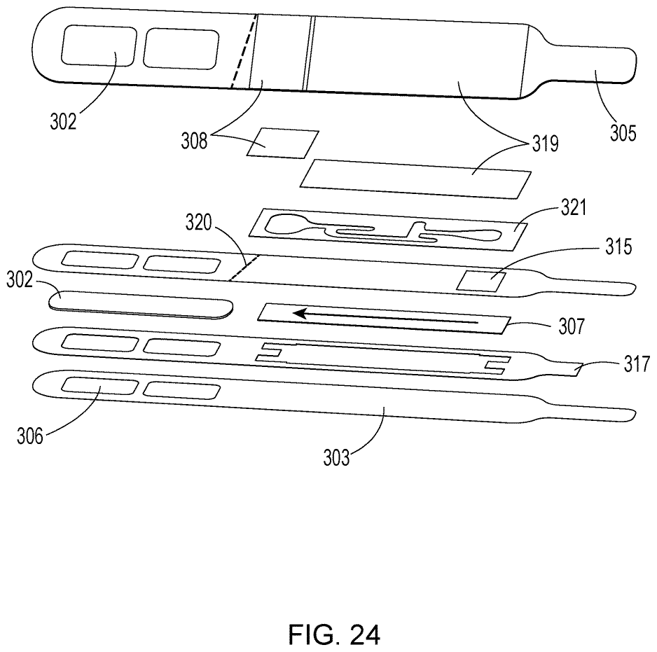

Another embodiment of the plasma strip shown in FIG. 24 includes an inertial filter (321) that uses fluidic channels to assist in pre-filtering the whole blood before it enters the input port (315) to the collection paper (307). By including multiple fluid traps and bends, RBCs become trapped while allowing the plasma to wick across the path. Channels widths can range from microns to millimeters. A TE pre-filter (308) is also included at the entrance of the inertial filter (321) to reduce the quantity of RBCs that the inertial filter (321) must remove.

After the fluid passes through the inertial filter (321), it enters the input port (315) and flows across the plasma separation and collection paper (307). After collection is ended, to allow access to the paper and to facilitate dry down, the plastic film, (with the attached inertial filter, TE membrane, and plastic cover) is torn off at the perforations (320) and is thrown away.

FIG. 24 shows DMS or DBS collection paper (302), pull tab (305), punch holes (306), base film 303, plasma separation and collection paper (307), track-etch pre-filter (308), input port (315), double sided tape (317), plastic film (inertial pre-filter) (319), tear perforation (320) and inertial pre-filter (321).

### Cell Collection Devices

Cell Collection by cell strip: Unlike the strips that are designed to collect samples that will be desiccated, such as DMS, DMPS, DBS, and/or DPS strips, the cell strip is not designed to deliver collected samples to the laboratory in a dried/desiccated state. The goal for the cell strip is collecting live cells and returning these cells to the laboratory in an unchanged intact state for analysis. For this, the collected sample must be kept hydrated.

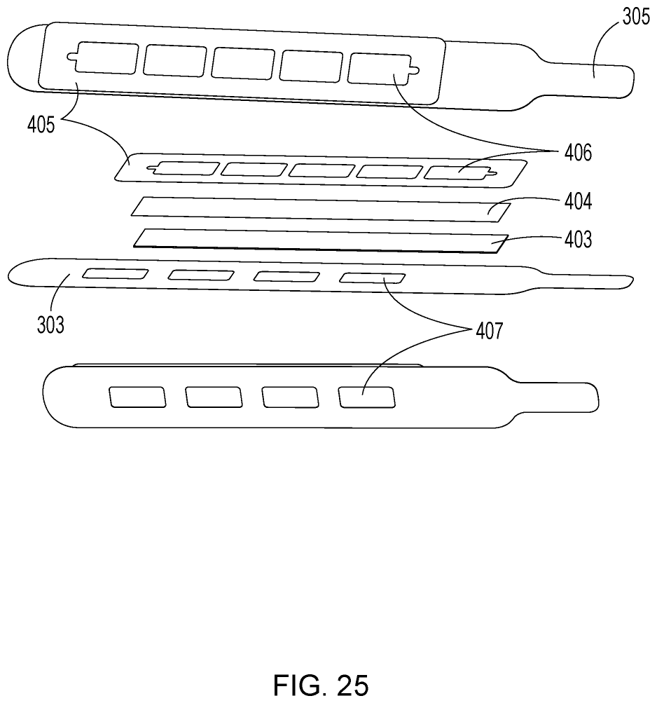

FIG. 25 illustrates a strip-based version of a cell collector designed to slide into the strip pocket (104). Similar to other strip designs, the cell collecting strip has a base film (303) with a pull tab (305) feature. The collection layers are sandwiched between the base film (303) and the adhesive frame (405). There are holes punched in the bottom film to allow fluid to flow through the strip (407).

While fluid is flowing through the strip, cell material is trapped in the wicking layer (404) and on the collection mesh (403). The wicking layer (404) is a hydrophilic material such as a foam or a felt and the mesh (403) is optimized to collect the target cell size, with pore sizes ranging from 10 um to 100 um. Multiple input ports (406) are punched into the adhesive frame to maximize collection area.

FIG. 25 shows pull tab (305), base film (303), collection mesh (403), wicking layer (404), adhesive frame 405, input ports 406 and through-flow ports (407).

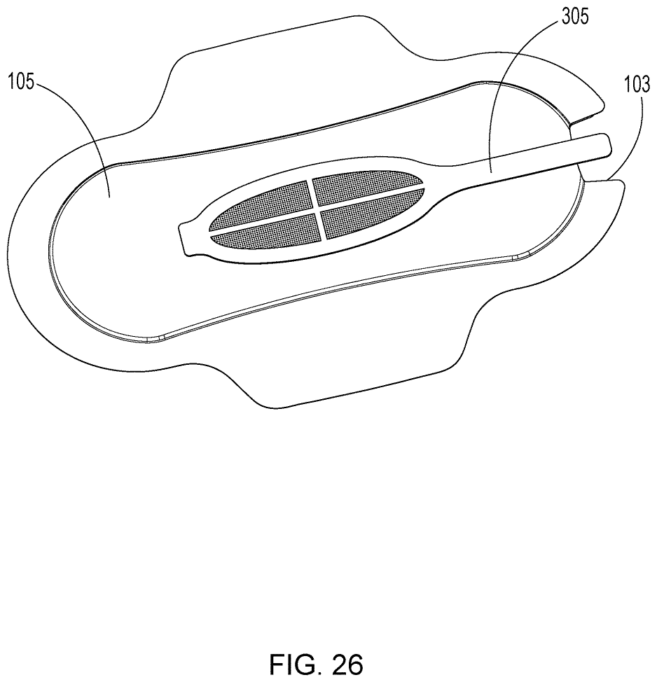

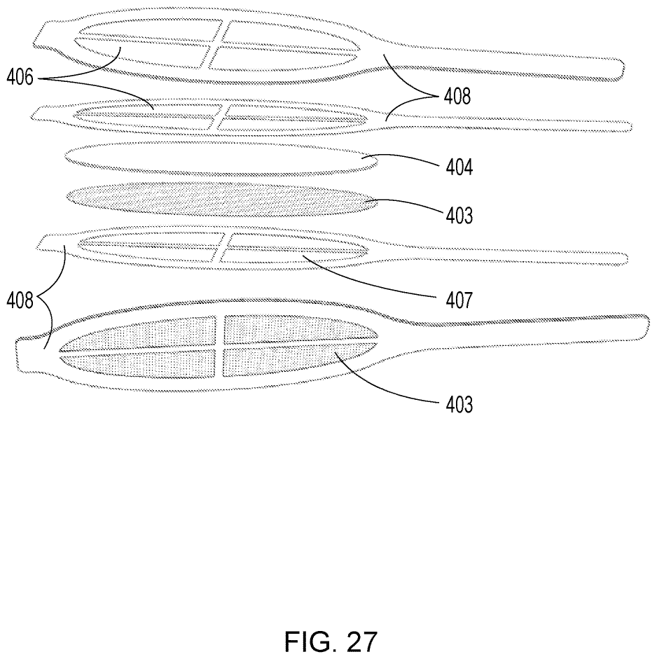

Cell collection by patch: FIGS. 26 and 27 illustrate a patch type: cell collector. The patch with a pull tab (305) attaches to the surface of the nonwoven top sheet (105) of the pad. This allows it to directly capture menstrual flow without the menses being absorbed and/or filtered by the non-woven top layer.

As seen in these figures, frame (408) surfaces and edges of the patch are in contact with the woman, therefore they must be constructed using soft compliant materials that will not irritate the user. The entrapment layers are similar to the strip version (401) of the cell collector with a wicking layer (404) above and mesh layer (403) under the wicking layer. This embodiment can be used on a off-the-shelf menstrual pad, where it can be adhered as a patch. If a notched pad is used, as described, the patch pull tap (305) extends to the pad notch (103) so it stays free of blood. The patch covers much of the pad surface with multiple input ports (406). The collection layers trap cells to be processed at the lab with most of the liquid soaking through to the pad so sample collection time can be extended.

FIG. 26 shows notch (103), pull tab (305) and the nonwoven surface of pad (105). FIG. 27 is an exploded view of the patch collector showing input ports (406), collection mesh (403), wicking layer (404) through flow ports (407) and frames (408).

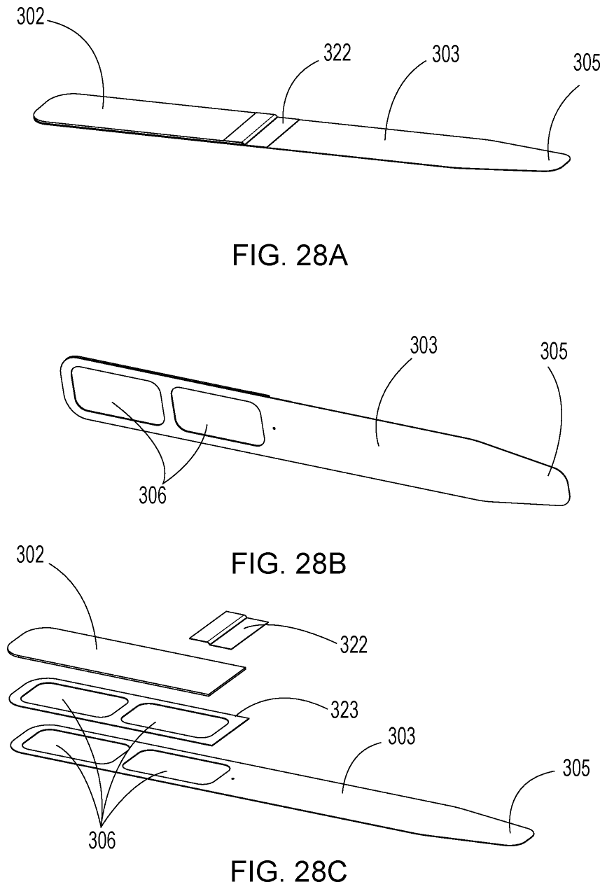

Another embodiment of a DMS or DBS collection strip (with double sided tape or a transfer adhesive) is illustrated in FIGS. 28A, 28B and 28C. In this embodiment, the DMS or DBS collection strip is fabricated using a double sided (DS) tape. The DS tape adheres the collection paper to the support film facilitating efficient manufacturing, optimal size, and maximized collection area.

The strip is constructed using a plastic film (303) (materials such as PET, PP, etc), a double-sided tape (323), and a collection paper (302). The DS tape (323) is sandwiched between the collection paper and plastic base film (303). The strip may or may not also use a narrow tape film to protect the leading edge of the collection paper to prevent delamination (322). The plastic film (303) and DS tape (323) have zones punched out to facilitate processing of the strip in the laboratory (306). The front of the strip narrows and functions as the pull tap (305) that the user engages with to remove the strip from the pad.

FIG. 28A shows collection filter paper (302), base film (303), pull tap (305) and tape to protect the edge of the collection paper (322). FIG. 28B shows base film (303), pull tab (305), and punch holes (306). FIG. 28C shows collection filter paper (302), base film (303), pull tab (305), punch holes (306) and protective tape (322).

Another aspect of the present invention relates to a method of high-speed manufacturing of an absorbent article such as feminine hygiene pads, urinary pads and the like that contain a strip for the purposes of collecting bodily fluids for analysis. More specifically the method provides a stable opening into one or more end seals of an absorbent article for easy insertion of a semi rigid component such as a fluid collection strip used for analyzing bodily fluids.

As noted previously, the collection device, such as a semi rigid strip, is typically inserted through a slit in the top face sheet or through a gap in the end seal. The end seal is typically made with an adhesive and/or mechanical crimp. In the prior art, a section of the end seal is left unsealed to facilitate an opening for the insertion of a strip. However, that results in a difficult and slow process due to the high drapability or limpness of the end seal. The end seal is typically made to be very soft and pliable for user comfort.

For high-speed manufacturing, a better process is required. The method of the present invention overcomes that issue by providing zone coating the absorbent pad with a hot melt adhesive to create an impervious zone around a pervious pocket and a notch cut that exposes said pocket. With this notch, a small section of the absorbent core is die cut away, being the approximate width of the of the pocket. This provides rigidity to the opening making it easier to insert the strip into the pocket.

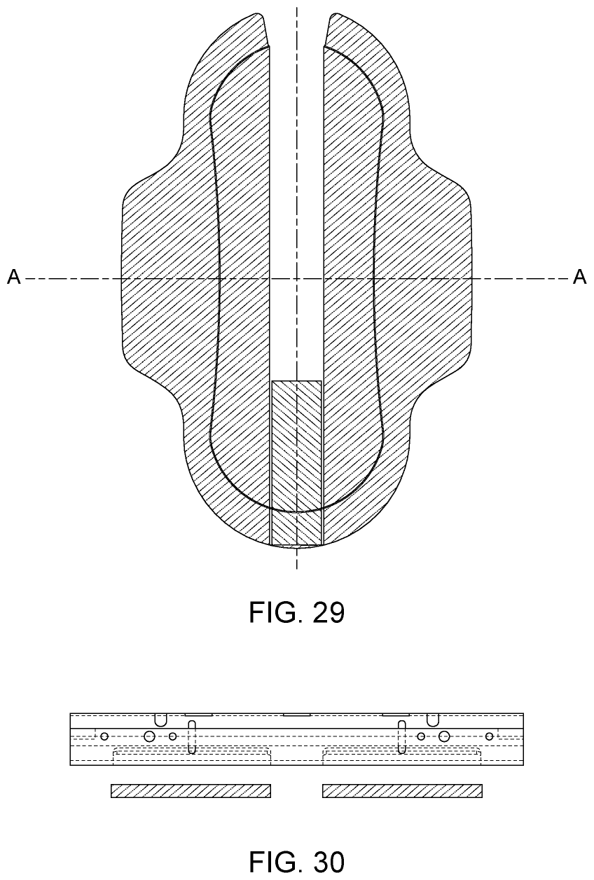

FIG. 29 shows a top view of the pad during the manufacturing process with the top layer removed showing the pocket created by the adhesive coating zones. FIG. 30 shows a structural view of the hot melt adhesive slot coating nozzle and a sectional view of the pad. A pocket is created in the pad for placement of the collection strip by zone coating the construction adhesive that is used to secure the nonwoven to the pad. This is accomplished using a slot coater with three solenoid-controlled application zones.

The left and right solenoids are kept open while the pad machine is running. This creates two areas of complete adhesive coverage. The center solenoid is cycled on and off to create an area of no adhesive which is the pocket for the strip. The length of the pocket is controlled by the length of time the solenoid is closed.

The nonwoven facing material is preferably a carded hydroentangled cotton. This process does not use any chemical binders to create the nonwoven structure which yields a soft hand web that is slightly fuzzy. The adhesive added to this nonwoven was sufficiently low as to allow cotton fibers to pierce the adhesive film creating points of entry for fluid to pass through the adhesive film into the absorbent core of the pad. To demonstrate this, a blue food coloring aqueous solution can be dispensed onto sections of a pad with an eye dropper.

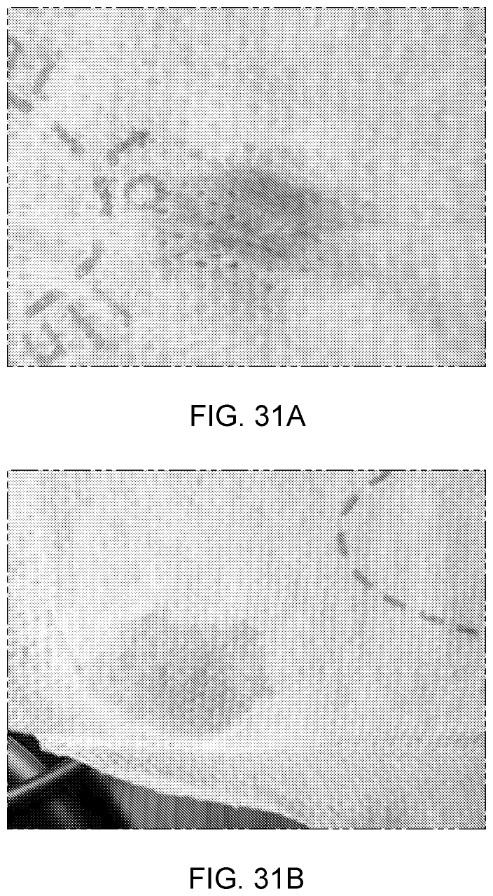

FIG. 31A shows a portion of the pad pocket and the fluid being absorbed directly into the absorbent layer. 31B shows a portion of the pad outside of the pocket with adhesive zones that provide barriers to selectively reduce the absorption rate into the fluid absorbing layer of the pad.

While multiple preferred embodiments of the present invention have been disclosed for purposes of illustration, it is obvious that many modifications and variations could be made thereto. It is intended to cover all of those modifications and variations which fall within the scope of the present invention, as defined by the following claims.

1-20. (canceled)

21. An absorbent pad having a peripheral edge, said pad comprising a fluid pervious top face sheet, a fluid absorbing layer, a fluid impervious back sheet, a pocket formed between said top sheet and said absorbing layer, and a fluid collection device adapted to be received in said pocket to collect bodily fluids for analysis, said pad comprising an indicator or indicators printed on said top face sheet centered under the vaginal canal, said indicator or indicators can be sized such that the time it takes the user to fill the shape corresponds to the time it takes to complete the fluid collection by said fluid collection device.

22. The absorbent pad of claim 21 wherein said collection device comprises a body and a pull tab extending from said collection device body, and a notch extending inwardly from said peripheral pad edge such that said pull tab of said collection device is exposed.

23. The absorbent pad of claim 22 further comprising a direction indicator on said top face sheet proximate said notch adapted to inform the user of the correct directional position of the pad in use.

24. The absorbent pad of claim 21 comprising a collection strip for use in said absorbent pad or to dose directly independent of said absorbent pad to collect a sample of menstrual, venous, capillary, or urinary origin comprising a single collection paper, a thin plastic base film and a bottom frame, wherein said collection paper is mounted to said thin plastic base film on said bottom frame and wherein said bottom frame comprises a single sided medical tape coated with an inert adhesive.

25. The absorbent pad of claim 21 comprising a collection strip for use in said absorbent pad or to dose directly independent of an absorbent pad to collect and process a sample of menstrual, venous, capillary, or urinary origin that incorporates a filter to separate red blood cells from whole blood to generate plasma as a result, said collection strip comprising a collection paper and a filter stack, wherein blood flows through said filter stack directly into said collection paper such that said filter stack provides filtration of the blood and isolates said collection paper from the unfiltered blood.

26. The absorbent pad of claim 21 comprising a plasma collection strip comprising a base film and a filter comprising a single layer of track-etch membrane and a collection paper situated between said membrane and said base film.

27. The absorbent pad of claim 26 wherein said plasma collection strip filter comprises a tortuous path depth filter mounted on a removable layer of medical tape.

28. The absorbent pad of claim 26 wherein said plasma collection strip filter comprises a lateral blood separating collection paper with an input port formed in a removable layer.

29. The absorbent pad of claim 26 wherein said plasma collection strip filter comprises a depth pre-filter incorporated into a removable layer and said track-etch membrane primary filter.

30. The absorbent pad of claim 26 herein said plasma collection strip filter comprises a track-etched membrane pre-filter incorporated into a removable layer and a lateral blood separating collection paper.

31. The absorbent pad of claim 26 wherein said plasma collection strip filter comprises a tortuous path depth pre-filter incorporated into a removable layer and a lateral blood separating collection paper.

32. The absorbent pad of claim 26 wherein said plasma collection strip filter incorporates a filter that uses channels shaped in tortuous paths formed in a sandwiched layer incorporated into a removable layer and a lateral blood separating collection paper

33. The absorbent pad of claim 26 wherein said plasma collection strip filter includes a track-etch pre-filter.

34. The absorbent pad of claim 26 wherein said plasma collection strip filter includes a track-etch post-filter.

35. The absorbent pad of claim 21 in combination with a container for transporting a fluid collection device of the type utilized in said absorbent pad to collect a sample, said container comprising a slot adapted to receive said fluid collection device, a desiccant, and a channel connecting said slot and said desiccant to allow moisture vapor transfer from said fluid collection device for dry-down of the sample during transit.

36. The container of claim 35 utilized to dose a sample, said container comprising a slot adapted to receive said fluid collection device, a desiccant, and a channel connecting said slot and said desiccant to allow moisture vapor transfer from said fluid collection device for dry-down of the sample during transit.

37. A cell collection patch for use in an absorbent pad for returning the cells to a laboratory in a hydrated state for DNA and slide based analysis, said patch comprising a frame with surfaces and edges in contact with the woman's vulva and epidermis, said surfaces constructed soft compliant materials, a wicking collection layer such as a foam, felt, a lattice structured material, or the like and a mesh layer, wherein said mesh layer is situated below said wicking layer, wherein said patch comprises multiple input ports and wherein said collection and mesh layers trap cells to be processed at the lab.

38. A cell collection strip system for use in an absorbent paid for collecting intact cell samples and returning the cells to the laboratory in a hydrated state, said system comprising a cell collection wicking medium such as foam, felt, a lattice structured material, or the like, a base film and an adhesive frame with said cell collection medium situated between said a base film and an adhesive frame, wherein said film comprises openings to allow fluid to flow through the strip, and a wicking medium layer comprising hydrophilic material for trapping cell material. as fluid is flowing through said cell collection strip.

39. The absorbent pad of claim 21 such as feminine hygiene pads and urinary pads, with a nonwoven facing material of a carded hydroentangled cotton, manufactured by a high-speed manufacturing method comprising the steps of:
creating a pocket in said pad to retain a fluid collection strip used for analyzing bodily fluids,
wherein said pocket is created by zone coating said pad with hot melt adhesive to secure the nonwoven facing material to the pad and to form a zone or zones of adjustable impermeability around said pocket,
wherein the zone coating is achieved by a hotmelt slot coating machine having left, right and center solenoid-controlled application zones;
wherein said left and right solenoids are kept open while the coating machine is running to create two distinct areas of adhesive coverage;
wherein said center solenoid of the coating machine is cycled on and off to create an area with no adhesive for the pocket of the strip; and
wherein the length of the pocket is controlled by the length of time said center solenoid of said machine is closed.

40. The method of claim 39 wherein the adhesive added to the nonwoven facing material by the machine is sufficiently low as to allow cotton fibers to pierce the adhesive film creating points of entry for fluid to pass through the adhesive film into the absorbent core of the pad.
